(19) United States
(12) Patent Application Publication
(10) Pub. No.: US 2021/0230525 A1
(43) Pub. Date: Jul. 29, 2021

(54) OOCYTE-HOLDING PIPETTE FOR SPERM INJECTION METHODS WITHOUT CYTOPLASMIC ASPIRATION

(71) Applicant: Francisco Vergara Alcaide, Gojar, Grenada (ES)

(72) Inventor: Francisco Vergara Alcaide, Gojar, Grenada (ES)

(21) Appl. No.: 17/052,306

(22) PCT Filed: May 3, 2019
(86) PCT No.: PCT/ES2019/070290

§ 371 Date: Nov. 2, 2020

(30) Foreign Application Priority Data
May 3, 2018 (ES) U201830624

(51) Int. Cl. C12M 3/00

(52) U.S. Cl. CPC C12M 21/06

(57) ABSTRACT

The invention relates to an oocyte-holding pipette for sperm injection methods without cytoplasmic aspiration, comprising an elongate, narrow, hollow, cylindrical body, inside which runs a suction conduit that communicates with a working end in order to hold an oocyte, said working end being flared to form a funnel defining an internal conical cavity, the proximal end of which, of smaller diameter, communicates with the suction conduit and the distal end of which, of larger diameter, is dimensioned to receive the oocyte in a tight-fitting manner, such that, upon aspirating same via the suction conduit, the oocyte remains partially trapped inside and becomes deformed, changing from its spherical shape in the resting state to an oval shape that tensions the surface and increases the turgor.

### OBJECT OF THE INVENTION

As expressed in the title of the present specification, the invention relates to an oocyte-holding pipette for sperm injection methods without cytoplasmic aspiration, providing the intended function thereof with advantages and novelty features that are described in detail below and constitute an alternative improvement in the current state of the art.

More specifically, the object of the invention focuses on a micropipette for use as means for holding an oocyte in intracytoplasmic sperm injection or ICSI methods, the novel structural configuration of which, unlike conventional pipettes, eschews the step of aspirating the cytoplasm with the injection needle to ensure the rupture of the cytoplasmic membrane, since the new pipette, provided with a flared end, holds the oocyte by suction, slightly deforming it inside said end, and said deformation causes an increase in oocyte turgor which allows breaking the cytoplasmic membrane with the injection pipette without first suctioning the cytoplasm.

### FIELD OF APPLICATION OF THE INVENTION

The field of application of the present invention is comprised within the sector of the industry dedicated to the manufacture of medical instruments, focusing particularly on the field of microdevices applicable for cellular-level procedures and interventions, and more specifically to devices intended for use in assisted reproduction procedures.

### BACKGROUND OF THE INVENTION

As is known, intracytoplasmic sperm injection or ICSI is an assisted reproduction technique consisting of the fertilization of oocytes by means of injecting a sperm into their cytoplasm using a micropipette, after obtaining and preparing the gametes for the purpose of obtaining embryos that can be transferred to the mother's womb.

To carry out said technique, the previously denuded oocyte must be held with a holding pipette, while the sperm is injected with another pipette, in this case an injection pipette.

The problem is that the membrane surrounding the oocyte is a hard-to-break membrane due to its high elasticity; in fact, the injection needle can be inserted into the cytoplasm without actually breaking the membrane (the membrane was microscopically invaginated by the injection pipette but did not break). The conventional way for breaking the membrane is to perform aspiration with the injection pipette until breaking the membrane. However, this leads to a disorganized cytoplasm that may critically affect the mitotic spindle (the location of which could only be estimated as it is not observed with routine ICSI technique) if its arrangement is altered during cytoplasmic aspiration.

The membrane must break for the proper release of the sperm into the cytoplasm, and also for the activation of the Ca++ or calcium ion channel mechanisms, which in turn intervene in the activation of cell division mechanisms.

To solve this problem, the applicant himself is the proprietor of utility model ES1187335U which describes a “pipette for retaining oocytes in intracytoplasmic sperm injection methods” the features of which allow holding the oocyte and performing injection through its membrane without prior cytoplasmic aspiration. Specifically, the configuration of said holding pipette has an innovative working end holding the oocyte by means of pressure, for which it has a decreasing frustoconical portion where the diameter of its cylindrical body is significantly reduced until defining a small support tip with a planar, straight, and angled configuration with respect to the cylindrical body.

Said holding pipette therefore holds the oocyte by means of a slight pressure, not by means of suction like conventional pipettes, which pressure causes an increase in oocyte turgor and thereby allows breaking the cytoplasmic membrane without having to aspirate the cytoplasm.

The objective of the present invention is to develop another different, alternative holding pipette model that does not require aspirating the cytoplasm with the injection pipette either, which model, unlike the one mentioned above, provides greater assurance when holding the oocyte, as this is an aspect of the pipette susceptible to improvement. Specifically, instead of acting by means of the working tip pressing on the oocyte, which may constitute a highly skilled operation so that the oocyte does not slide to either side when being pressed, the present invention acts by means of suctioning the oocyte with the holding pipette itself, just like conventional pipettes with the only difference being that while those (conventional) pipettes only perform aspiration to hold the oocyte without deforming it, this pipette (the pipette of the present invention) aspirates the oocyte to deform it while it is partially housed in the flared working end thereof, as will be explained in detail below.

Moreover, and as reference to the current state of the art, it must be indicated that the applicant at least is unaware of the existence of any other pipette with the same application for holding oocytes in intracytoplasmic sperm injection methods or another similar pipette having technical, structural, and constitutive features that are identical or similar to those of the pipette herein claimed.

### BRIEF DESCRIPTION OF THE INVENTION

The oocyte-holding pipette for sperm injection methods without cytoplasmic aspiration proposed by the invention is therefore configured as a remarkable novelty in its field of application, given that the objectives indicated above are specifically and satisfactorily achieved in view of its implementation, with the characterizing details making it possible and distinguishing same being suitably described in the final claims accompanying the present description.

Specifically, as described above, the invention proposes a pipette for holding oocytes in intracytoplasmic sperm injection methods which fulfills said purpose of holding without cytoplasmic aspiration, as it eschews such step of performing aspiration with the injection needle, which is required in conventional holding pipettes to ensure the rupture of the cytoplasmic membrane and cause the activation of the oocyte and to more readily deposit the sperm in the cytoplasm, but which, as pointed out in the preceding section, may damage the original structure of the cytoplasm and, in some cases, alter the structure of the mitotic spindle.

To that end, the holding pipette of the present invention comprises an elongate and narrow cylindrical body, the working end of which, i.e., the end used for holding the oocyte, is characterized by having a flared funnel-shaped configuration communicating through its smaller-diameter end with the aspiration conduit running along the cylindrical body, the larger opposite end being sized to receive the oocyte in a tight-fitting manner, such that, upon aspirating said oocyte, it remains trapped inside said flared end and becomes deformed enough so as to have a more turgid surface which facilitates the application of the injection pipette without having to previously aspirate the cytoplasm with the pipette.

Therefore, as with the conventional holding pipette, the pipette without cytoplasmic aspiration of the invention fulfills the purpose of holding the oocyte in ICSI methods but eschews the step of aspirating the cytoplasm with the injection needle, which is required in conventional holding pipettes having a rounded working end to ensure the rupture of the cytoplasmic membrane and cause the activation of the oocyte and to more readily deposit the sperm in the cytoplasm, but which damages the original structure of the cytoplasm and, in some cases, may alter the structure of the mitotic spindle.

Similarly to conventional holding pipettes, the holding pipette of the invention is manufactured from embryo-tested glass, specifically borosilicate, and the method for manufacturing the holding pipette of the invention is also similar to the method for manufacturing these conventional holding pipettes.

In turn, the dimensions of the pipette and its parts are conceived so that it can be used in the same microinjection equipment in which conventional holding pipettes are used, only slightly changing the ICSI technique protocol.

The described pipette therefore represents an innovation in structural and constitutive features that have been unknown up until now, and these reasons, combined with its practical usefulness, provide it with sufficient grounds to obtain the exclusive privilege that is sought.

### DESCRIPTION OF THE DRAWINGS

To complement the description that is being made and for the purpose of helping to better understand the features of the invention, a set of drawings is attached to the present specification as an integral part thereof in which the following is depicted in a non-limiting, illustrative manner:

FIG. 1 shows a side elevational view of the holding pipette object of the invention, with its general configuration and the main parts it comprises being observed.

FIG. 2 shows an enlarged view of detail A indicated in FIG. 1 and corresponding to the working end of the pipette, according to the invention, with the general configuration thereof being observed.

FIGS. 3 and 4 show respective enlarged views according to a longitudinal section view and an enlarged front elevational view, respectively, of the working end of the pipette shown in FIG. 2, with the configuration and dimensions thereof being observed in greater detail.

FIGS. 5 and 6 show respective schematic views of the mode of operation of the working end of the pipette of the invention for holding the oocyte by means of suction and deformation of the oocyte and without aspiration of its cytoplasm.

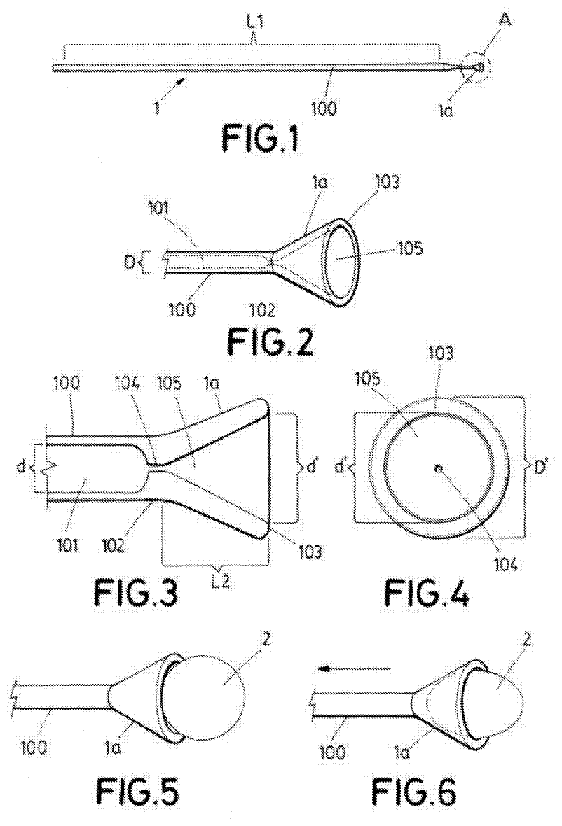

### PREFERRED EMBODIMENT OF THE INVENTION

In view of the mentioned figures and according to the numbering used, a non-limiting embodiment of the holding pipette of the invention can be seen therein, which pipette comprises the parts and elements indicated and described in detail below.

In this sense, as seen in said figures, the holding pipette (1) of the invention comprises an elongate, narrow, and hollow cylindrical body (100), inside which runs a suction conduit (101) that communicates with a working end (1a) for holding the oocyte (2), which end is characterized by having a flared funnel-shaped configuration defining an internal conical cavity (105), the smaller-diameter proximal end (102) of which communicates with the mentioned suction conduit (101), the larger-diameter distal end (103) being sized to receive the oocyte (2) in a tight-fitting manner, such that, upon aspirating same via suction conduit (101), the oocyte remains partially trapped inside and becomes deformed, as shown in FIGS. 5 and 6, changing from its spherical shape in the resting state to an oval shape that tensions the surface and determines a more turgid oocyte which facilitates the application of the injection pipette without having to previously aspirate the cytoplasm with the pipette.

In the preferred embodiment, the cylindrical body (100) has a length (L1) of about 5 cm and an external diameter (D) of 500 micra, and the suction conduit (101) has a constant internal diameter (d) of 80 micra along its entire length with the exception of the point at which it is joined to the funnel-shaped working end (1a), there being, in the outlet opening of said conduit (101), coinciding with the smaller-diameter proximal end (102) of said funnel, a constriction (104) where said diameter is reduced considerably to only about 17 micra.

Furthermore, the internal conical cavity (105) defining said funnel-shaped working end (1a) also preferably has a length (L2) of 128 micra and an internal diameter (d), at its larger-diameter distal end (103), of 120 micra, whereas the external diameter (D) of said distal end (103) of the working end (1a) is preferably 180 micra.

Having sufficiently described the nature of the present invention, as well as the manner of putting it into practice, it is not considered necessary to expand on this explanation so that one skilled in the art will understand its scope and the advantages derived from it, hereby stating that within its essential nature, the invention may be carried out to practice in other embodiments which differ in detail from that indicated by way of example and which will likewise attain the protection that is sought provided that its fundamental principle is not altered, changed, or modified.

1-5. (canceled)

6. Oocyte holding pipette for sperm injection procedures without cytoplasmic aspiration that, configured from a long, narrow and hollow cylindrical body, through which a suction conduit runs that communicates with a working end to hold an oocyte, is characterized in that said working end has a bell-shaped funnel-shaped configuration that defines an internal conical cavity, whose proximal end, of smaller diameter, communicates with the suction conduit and whose distal end, with a larger diameter, is dimensioned to tightly receive the oocyte in such a way that, when suction is applied to it through the suction conduit, is partially trapped inside to deform, from its spherical shape in a state of rest, to an oval shape that tightens its surface and increases its turgor.

7. Oocyte holding pipette for sperm injection procedures without cytoplasmic aspiration according to claim 6, characterized in that the cylindrical body is about 5 cm long and about 500 microns in external diameter, and the duct suction tube has about 80 microns of constant internal diameter throughout its length except for the point where it joins the working end in the shape of a funnel, existing at the outlet mouth of said conduit, coinciding with the proximal end of smaller diameter of said funnel, a constriction where its diameter is considerably reduced.

8. . Oocyte holding pipette for sperm injection procedures without cytoplasmic aspiration according to claim 7, characterized in that in the constriction the diameter of the duct is reduced to only about 17 microns.

9. Oocyte holding pipette for sperm injection procedures without cytoplasmic aspiration according to claim 6, characterized in that the internal conical cavity defining the working end in the shape of a funnel has a length of about 128 microns and an inner diameter, at its distal end with a larger diameter, of about 120 microns.

10. Oocyte holding pipette for sperm injection procedures without cytoplasmic aspiration according to claim 7, characterized in that the internal conical cavity defining the working end in the shape of a funnel has a length of about 128 microns and an inner diameter, at its distal end with a larger diameter, of about 120 microns.

11. Oocyte holding pipette for sperm injection procedures without cytoplasmic aspiration according to claim 8, characterized in that the internal conical cavity defining the working end in the shape of a funnel has a length of about 128 microns and an inner diameter, at its distal end with a larger diameter, of about 120 microns.

12. Oocyte holding pipette for sperm injection procedures without cytoplasmic aspiration according to claim 9, characterized in that the external diameter of the distal end of the funnel-shaped working end is about 180 microns.

13. Oocyte holding pipette for sperm injection procedures without cytoplasmic aspiration according to claim 10, characterized in that the external diameter of the distal end of the funnel-shaped working end is about 180 microns.

15. Oocyte holding pipette for sperm injection procedures without cytoplasmic aspiration according to claim 11, characterized in that the external diameter of the distal end of the funnel-shaped working end is about 180 microns.
